(19) United States
(12) Patent Application Publication
(10) Pub. No.: US 2014/0056835 A1
(43) Pub. Date: Feb. 27, 2014

(54) COSMETIC COMPOSITION FOR SKIN WHITENING COMPRISING SMALL MOLECULE-PEPTIDE CONJUGATE

(71) Applicant: AE KYUNG INDUSTRIAL CO., LTD., Seoul (KR)

(72) Inventors: In Shik CHO, Seoul (KR); Hyung Seo GOO, Daejon (KR); Gyeong Yup CHI, Daejon (KR); Han Young KIM, Daejon (KR); Hye Suk LEE, Daejon (KR)

(73) Assignee: AE KYUNG INDUSTRIAL CO., LTD., Seoul (KR)

(21) Appl. No.: 13/973,099

(22) Filed: Aug. 22, 2013

(30) Foreign Application Priority Data
Aug. 24, 2012 (KR) 10-2012-0093278
Aug. 24, 2012 (KR) 10-2012-0093287

(51) Int. Cl. C07C 237/22

(52) U.S. Cl. CPC C07C 237/22
USPC 424/62; 564/153

(57) ABSTRACT

The present invention relates to a cosmetic composition for skin whitening comprising a small molecule-peptide conjugate. More specifically, it concerns the cosmetic composition for skin whitening comprising Caffeic acid-LG-NH₂ or Coumaric acid-LG-NH₂ as an active ingredient, which inhibits tyrosinase activity and melanin production, thereby showing an excellent skin whitening effect, and having no side effects to the skin by using a peptide component.

### FIELD OF THE INVENTION

The present invention relates to a cosmetic composition for skin whitening comprising a small molecule-peptide conjugate. More specifically, it concerns the cosmetic composition for skin whitening comprising Caffeic acid-LG-NH₂ or Coumaric acid-LG-NH₂ as an active ingredient, which inhibits tyrosinase activity and melanin production, thereby showing an excellent skin whitening effect, and having no side effects to the skin by using a peptide component.

### BACKGROUND OF THE INVENTION

One of the most important factors in skin whitening is the production of melanin in the skin melanocytes.

Melanin production involves tyrosine, which is converted to dopa, then to dopaquinone by the catalytic action of tyrosinase existing inside the melanocyte, and further intermediate products such as dopachrome. Melanin exists in the skin and protects the body from UV rays and such, and has an important function in the regulation of internal secretion of hormones. However, hyperpigmentation is known to cause formation of age spots, uneven color, freckles, and melasma and acceleration of skin aging, and is also known to have an important function in the induction of skin cancers. As a result, there has been active research and development related to the prevention of hyperpigmentation.

Substances such as vitamin C, mulberry root extracts, kojic acid, arbutin, licorice extracts, and hydroquinone are known to be effective for skin whitening. Kojic acid has been reported to be a carcinogenic substance and banned for use as a cosmetic component. Hydroquinone has toxicity against melanocytes, and in vivo test results indicate that it is a carcinogenic substance and poses a risk for permanent depigmentation; hence, importing hydroquinone into the country as a cosmetic component is currently prohibited.

Despite their effectiveness in skin whitening, the said chemical substances have limitation in their use as they accompany problems of toxicity or side effects to the skin. Therefore, in recent years, there has been active research for developing naturally derived, biocompatible skin-whitening components that could minimize side effects to the skin.

The inventors hence repeated studies to develop a material from naturally derived substances and peptides so that it has an excellent skin whitening effect while ensuring safety and minimizing side effects. As a result, the inventors invented a cosmetic composition for skin whitening comprising Caffeic acid-LG-NH₂ or Coumaric acid-LG-NH₂ as an active ingredient, thereby showing excellent skin whitening effect, and also having no side effects to the skin by using a peptide component.

### DESCRIPTION OF THE INVENTION

### Technical Problems

One aspect of the present invention is to provide a cosmetic composition that has an excellent inhibitory effect on tyrosinase activity.

Another aspect of the present invention is to provide a cosmetic composition that has an excellent inhibitory effect on melanin production.

Another aspect of the present invention is to provide a cosmetic composition that has an excellent skin whitening effect.

Another aspect of the present invention is to provide a cosmetic composition for skin whitening that is safe to the human skin and has no side effects resulting from prolonged use.

### Technical Solutions

The present invention provides a cosmetic composition for skin whitening comprising, as an active ingredient, chemical compounds expressed in the following Formula 1:

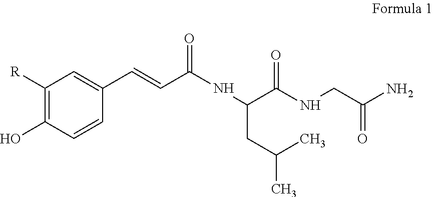

wherein R is H or OH.

Henceforward, the formula is expressed as Coumaric acid-LG-NH₂ when R is H, and expressed as Caffeic acid-LG-NH₂ when R is OH.

In an embodiment of the present invention, the aforementioned Caffeic acid-LG-NH₂ or Coumaric acid-LG-NH₂ is contained 0.000001 to 30% by weight with respect to the total weight of the cosmetic composition.

In another embodiment, the formulation of the aforementioned skin-whitening cosmetic composition is for external use.

In another embodiment, the formulation of the aforementioned skin-whitening cosmetic composition is selected from a group consisting of skin lotions, gels, aqueous liquids, creams, essences, oil-in-water types, water-in-oil types, ointments, and a combination thereof.

In another embodiment, the aforementioned skin-whitening cosmetic composition further comprises an additive selected from a group consisting of oil, water, surfactants, moisturizers, lower alcohols, thickeners, chelation agents, colorants, preservatives, perfumes, and a mixture thereof.

### Advantageous Effects

The composition of the present invention is effective in skin whitening due to its inhibitory effect on tyrosinase activity and melanin production. In addition, the composition of the present invention causes minimal skin irritation, and is safe to the human skin with no side effects resulting from prolonged use since it contains the peptide component. Furthermore, a variety of formulations is possible with the composition of the present invention.

### BRIEF DESCRIPTION OF THE DRAWINGS

FIG. 1 is a graph that compares cell survival rates when Caffeic acid-LG-NH₂ and arbutin are applied according to Experimental Example 3.

FIG. 2 is a graph that compares cell survival rates when Coumaric acid-LG-NH₂ and arbutin are applied according to Experimental Example 3.

### DETAILED DESCRIPTION OF THE INVENTION

The cosmetic composition for skin whitening according to the present invention is characterized by comprising chemical compounds as described below:

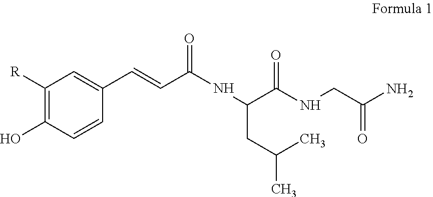

wherein R is H or OH.

In the above Formula 1, Coumaric acid-LG-NH₂ where R═H and Caffeic acid-LG-NH₂ where R═OH are designed to have high biocompatibility by comprising a peptide, and to enhance efficacy of the peptide material by introducing a coumaric acid or a caffeic acid to the N-terminal of the peptide.

The chemical compounds expressed in the above Formula 1 are prepared via coupling of a peptide with a coumaric acid or a caffeic acid.

The peptide of the present invention has a dipeptide form in which Leucine (Leu, L)-Glycine (Gly, G) is linked via an amide bond. Also, the OH group in the C-terminal portion is substituted with NH₂.

In an embodiment of the present invention, introducing the basic peptide structure and organic molecule of the aforementioned chemical compounds can be carried out with a solid-phase synthesis method.

The Coumaric acid-LG-NH₂ and Caffeic acid-LG-NH₂ as shown above can be contained 0.000001 to 30% by weight, more specifically 0.001 to 10% by weight, with respect to the total weight of the cosmetic composition. The skin whitening effect may be insignificant when the aforementioned chemical compounds are contained less than 0.000001% by weight, while the stability of formulation may decline when contained greater than 30% by weight.

Furthermore, besides the ingredients specified above, the skin-whitening cosmetic composition of the present invention can be appropriately mixed with general cosmetic additives such as oil, water, surfactants, moisturizers, lower alcohols, thickeners, chelation agents, colorants, preservatives, or perfumes, as needed for its respective formulations.

The formulation of the skin-whitening cosmetic composition containing the aforementioned Coumaric acid-LG-NH₂ or Caffeic acid-LG-NH₂ has no special constraints and can be adequately selected according to one's purpose. In an embodiment of the present invention, the formulation of the aforementioned skin-whitening cosmetic composition is for external use. For example, the skin-whitening cosmetic composition of the current invention can be prepared for a formulation of skin lotions, gels, aqueous liquids, creams, essences, oil-in-water or water-in-oil types, ointments, and so on. More specifically, the skin-whitening cosmetic composition of the present invention can be prepared for a formulation of skin ointments for external use, skin toners, nutrient lotions, nutrient creams, massage creams, face packs, emulsions, oil gels, and so on.

Hereinafter, the present invention is described in detail through preparation examples, experimental examples, and formulation examples. These are only intended for describing the present invention and do not limit the scope of the invention.

### Preparation Example 1

### Preparation of Caffeic Acid-LG-NH₂

1 g of Rink amide resin (substitution rate: 0.5 mmol/g) was added to a reactor and swollen in N-methylpyrrolidone (NMP) solvent for 2 hours. Then, 20% piperidine/NMP was added for a 30-minute reaction so that the Fmoc protecting group of the resin was removed. After removing the solution, 20% piperidine/NMP was added again to the resin-containing reactor and left for reaction for 1 hour. After removing the solution once more, the resin was washed with NMP, methanol, and Dichloromethane (DCM) for 3 times each, and again with NMP for the last time to remove any remaining reagent solution.

297 mg of Fmoc-Gly-OH (2 equivalents of the substituent), 442 mg of BOP reagent (1 equivalent of the amino acid reagent), 135 mg of 1-Hydroxybenzotriazole (HOBt) (1 equivalent of the amino acid reagent), and 142 mg of diisopropylethylamine (DIEA) (1.1 equivalents of the amino acid reagent) were added to the reactor with 10-ml NMP then left for a 3-hour reaction. Termination of the reaction was confirmed with Kaiser's ninhydrin test (Anal. Biochem., 1981, 117, 147).

After removing all the solution from the reactor, the resin was washed with NMP, methanol, and DCM for 3 times each, and again with NMP for the last time. To the fully washed resin, 20% piperidine/NMP was added, and the Fmoc protecting group was removed using the identical reaction procedure as described earlier. Afterwards, Fmoc-Leu-OH was introduced to the resin using the same method.

180 mg of caffeic acid (2 equivalents of the substituent), 442 mg of BOP reagent (1 equivalent of the amino acid reagent), 135 mg of 1-HOBt (1 equivalent of the amino acid reagent), 142 mg of DIEA (1.1 equivalents of the amino acid reagent), and 10-ml NMP were added to the resin introduced with Leucine (Leu, L)-Glycine (Gly, G), and left for a 24-hour reaction. Termination of the reaction was confirmed with Kaiser's ninhydrin test.

Caffeic acid-Leu-Gly-NH₂, prepared on Rink amide resin as above, was separated from the resin using reagent K by a known method (Synthetic Peptides: A User's Guide (G. A. Grant, ed.), W.H. Freeman and Company, New York, 1992). The separated small molecule-peptide conjugate was precipitated using cold ether, and then centrifuged at 0° C. to be separated from the solution. The procedure of adding 20-ml cold ether and centrifugation in the identical condition (as above) was repeated for 3 times so that impurities were removed from the separated peptide derivative. The final product was obtained after the centrifuged peptide derivative was freeze-dried for 24 hours.

### Preparation Example 2

### Preparation of Coumaric acid-LG-NH₂

1 g of Rink amide resin (substitution rate: 0.5 mmol/g) was added to a reactor and swollen in N-methylpyrrolidone (NMP) solvent for 2 hours. Then, 20% piperidine/NMP is added for a 30-minute reaction so that the Fmoc protecting group of the resin was removed. After removing the solution, 20% piperidine/NMP was added again to the resin-containing reactor and left for reaction for 1 hour. After removing the solution once more, the resin was washed with NMP, methanol, and Dichloromethane (DCM) for 3 times each, and again with NMP for the last time to remove any remaining reagent solution.

297 mg of Fmoc-Gly-OH (2 equivalents of the substituent), 442 mg of BOP reagent (1 equivalent of the amino acid reagent), 135 mg of 1-Hydroxybenzotriazole (HOBt) (1 equivalent of the amino acid reagent), and 142 mg of diisopropylethylamine (DIEA) (1.1 equivalents of the amino acid reagent) were added to the reactor with 10-ml NMP then left for a 3-hour reaction. Termination of the reaction was confirmed with Kaiser's ninhydrin test (Anal. Biochem., 1981, 117, 147).

After removing all the solution from the reactor, the resin was washed with NMP, methanol, and DCM for 3 times each, and again with NMP for the last time. To the fully washed resin, 20% piperidine/NMP was added, and the Fmoc protecting group was removed using the identical reaction procedure as described earlier. Afterwards, Fmoc-Leu-OH was introduced to the resin using the same method.

180 mg of coumaric acid (2 equivalents of the substituent), 442 mg of BOP reagent (1 equivalent of the amino acid reagent), 135 mg of 1-HOBt (1 equivalent of the amino acid reagent), 142 mg of DIEA (1.1 equivalents of the amino acid reagent), and 10-ml NMP were added to the resin introduced with Leucine (Leu, L)-Glycine (Gly, G), and left for a 24-hour reaction. Termination of the reaction was confirmed with Kaiser's ninhydrin test.

Coumaric acid-Leu-Gly-NH₂, prepared on Rink amide resin as above, was separated from the resin using reagent K by a known method (Synthetic Peptides: A User's Guide (G. A. Grant, ed.), W.H. Freeman and Company, New York, 1992). The separated small molecule-peptide conjugate was precipitated using cold ether, and then centrifuged at 0° C. to be separated from the solution. The procedure of adding 20-ml cold ether and centrifugation in the identical condition (as above) was repeated for 3 times so that impurities were removed from the separated peptide derivative. The final product was obtained after the centrifuged peptide derivative was freeze-dried for 24 hours.

### Experimental Example 1

### Test for Inhibitory Effect on Tyrosinase Activity

When tyrosinase gets activated by the influence of UV rays, surrounding environment, or internal hormones, tyrosine or DOPA (dioxyphenylalanin) goes through a dopachrome production pathway and transforms into a melanin via autoxidation reaction.

Through this experiment, degree of inhibition on melanin pigmentation was determined by measuring the presence of dopachrome, which is an intermediate product.

Tyrosinase used was purchased from Sigma and had been separated from mushrooms and purified. 50 ul of test samples at concentrations of 100 uM and 50 uM were placed in a 96-well plate. Afterwards, 50 ul of 2400 units/ml tyrosinase solution and 100 ul of 0.3 mg/ml L-tyrosine solution were added in sequence. Instead of the test samples, controls had the same amounts of solvent that dissolved the samples. Following a 10-minute reaction at 37° C., absorbance was measured at 475 nm using an ELISA reader.

Tyrosinase activity inhibition rate of each sample solution was calculated according to the following Equation 1, and the results are shown in Table 1.

Tyrosinase Activity Inhibition Rate (%)={(A−B)−(C−D)}/(A−B)×100 Equation 1

A: absorbance of control

B: blank absorbance of control

C: absorbance of sample

D: blank absorbance of sample

TABLE 1

| | Tyrosinase activity inhibition rate (%) |
|---|---|
| Caffeic acid-LG-NH₂ 100 uM | 26.8 |
| Caffeic acid-LG-NH₂ 50 uM | 24.2 |
| Coumaric acid-LG-NH₂ 100 uM | 34.8 |
| Coumaric acid-LG-NH₂ 50 uM | 25.1 |
| Caffeic acid 100 uM | 21.3 |
| Caffeic acid 50 uM | 14.8 |
| Coumaric acid 100 uM | 24.6 |
| Coumaric acid 50 uM | 21.3 |
| Arbutin 100 uM | 14.4 |
| Arbutin 50 uM | 9.2 |

As shown in Table 1, tyrosinase activity inhibition rates of Coumaric acid-LG-NH₂ and Caffeic acid-LG-NH₂ were superior to those of a comparative substance, arbutin. Based on these results, the skin-whitening cosmetic composition of the present invention can be expected to bring skin-whitening effect through an effective inhibition of tyrosinase activity.

### Experimental Example 2

### Test for Inhibitory Effect on Melanin Production

Inhibitory effect on melanin formation was measured using B16F1 melanoma cells. B16F1 melanoma cells were inoculated into a 6-well plate at a density of 3×10⁵ (cells/well) with DMEM containing 10% FBS. Cells were incubated for 1 day in 5% CO₂ at 37° C. Then the medium was replaced with new DMEM, and samples were prepared at 50-uM concentration and incubated for 2 days. After 2 days, the medium was removed and the cells were washed with PBS (phosphate buffered saline), treated with trypsin, and then retrieved. The retrieved cells were centrifuged and the supernatant was discarded. Intracellular melanin was obtained by treating the cells with 0.2 ml of 1N NaOH+10% DMSO solution. The collected cells were transferred to a 96-well plate and absorbance was measured at 490 nm. The amount of intracellular melanin was estimated using standard curve with synthetic melanin as a standard substance. The amount of protein was quantified via absorbance measurement using a protein assay reagent. For the amount of melanin produced, the amount of melanin for a given protein was estimated. Arbutin at 50 uM was used as a control, and for a negative control, the reaction solution without samples was used. Table 2 shows results from the test for inhibitory effect on melanin production in negative controls.

TABLE 2

| | Melanin production inhibition rate (%) |
|---|---|
| Caffeic acid-LG-NH₂ 50 uM | 41 |
| Coumaric acid-LG-NH₂ 50 uM | 45.3 |
| Caffeic acid 50 uM | 16 |
| Coumaric acid 50 uM | 23 |
| Arbutin 50 uM | 18 |

It was confirmed from the above results that Caffeic acid-LG-NH₂ and Coumaric acid-LG-NH₂ had excellent inhibitory effects on melanin production. Based on these results, the skin-whitening cosmetic composition of the present invention can be expected to bring skin-whitening effect through an effective inhibition of melanin production.

### Experimental Example 3

### Test for Cytotoxicity

HaCaT (human keratinocytes) were used for measuring the degree of cytotoxicity. HaCaT cells were inoculated into a 96-well plate at a density of 6×10³ (cells/well) with DMEM containing 10% FBS. Cells were incubated for 1 day in 5% CO₂ at 37° C. Then the medium was replaced with new DMEM, and samples were treated using a continuous dilution method starting at a concentration of 2.5 mM and then incubated for 1 day. After 1 day, the medium was treated with 10 ul of MTT reagent at 5 mg/ml and incubated for 4 hours in 5% CO₂ at 37° C. After removing the medium, 150 ul of DMSO reagent was added to dissolve the purple crystals, and absorbance was measured at 540 nm.

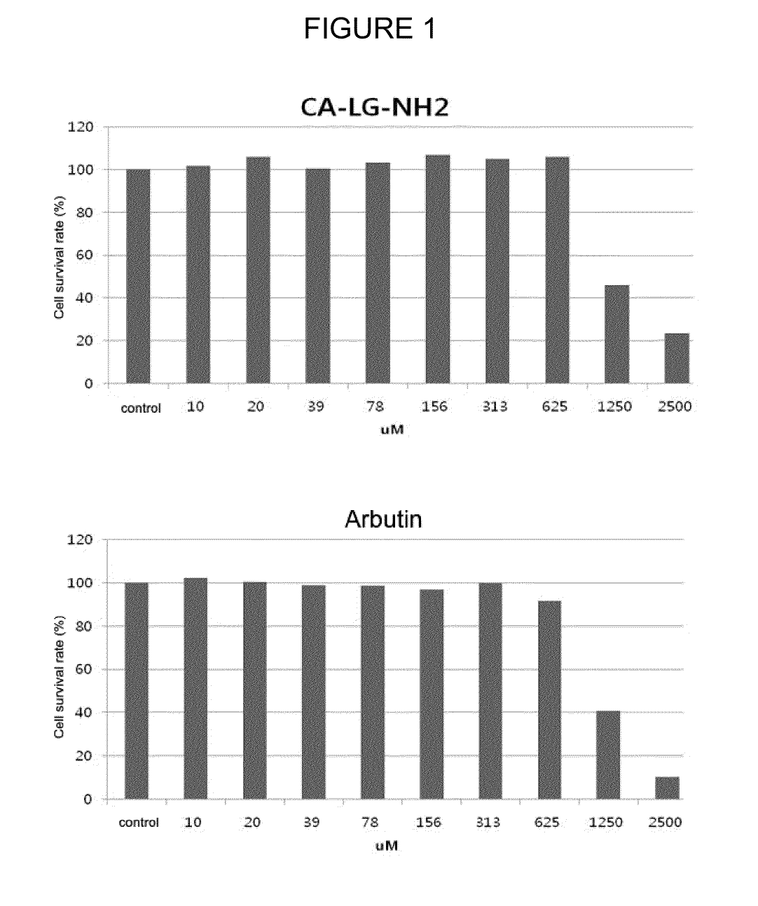

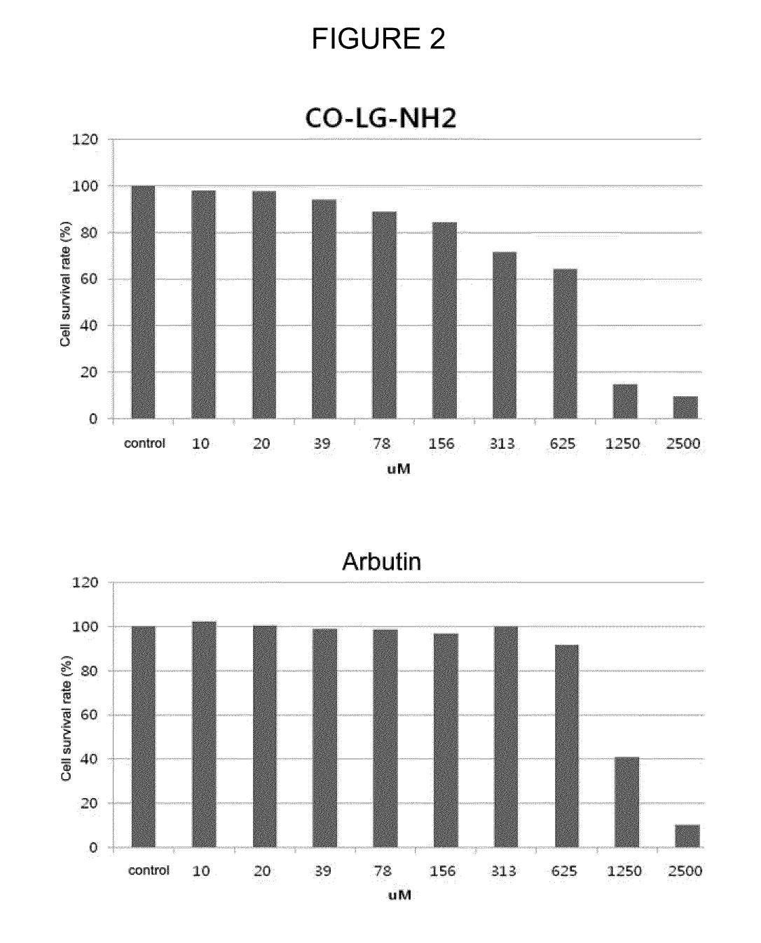

For a negative control, the reaction solution without samples was used. Also, a comparison was made with a conventional skin-whitening agent, arbutin. Cell survival rate in each sample solution was calculated according to the following Equation 2. Results of the measurement are shown in FIGS. 1 and 2.

Cell Survival Rate (%)=A/B×100 Equation 2

A: absorbance of sample

B: absorbance of negative control

The results in FIGS. 1 and 2 confirmed that Coumaric acid-LG-NH₂ and Caffeic acid-LG-NH₂ had very low cytotoxicity. Based on these results, the skin-whitening cosmetic composition of the present invention is considered to be safe to the skin.

### Experimental Example 4

### Test for Skin Irritation

A human patch test was conducted to assess the degree of skin irritation. Twenty normal subjects over 20 years of age participated in the human patch test. Samples were prepared into 0.001%, 0.01%, 0.1%, 1%, and 10% aqueous solutions, and then attached to an inner upper arm for 48 hours. Following removal, an initial read was made. When 72 hours had elapsed, a second read was made to determine results of skin response. Valuation criteria and calculation for the irritation index are as follows, and the results from each sample are shown in Tables 3 and 4, respectively.

| Response | Weighted value | Valuation criterion |
|---|---|---|
| − | 0.0 | No response |
| ± | 0.5 | Questionable positive response |
| + | 1.0 | Weak positive response |
| ++ | 2.0 | Strong positive response |
| +++ | 3.0 | Extreme positive response |

Mean score=Σ(weighted value×number of subjects showing positive reaction)/(maximum weighted value×total number of subjects)×100 Equation 3

TABLE 3

| | Mean score | Degree of irritation |
|---|---|---|
| Caffeic acid-LG-NH₂ 0.001% | 0.0 | None |
| Caffeic acid-LG-NH₂ 0.01% | 0.0 | None |
| Caffeic acid-LG-NH₂ 0.1% | 0.0 | None |
| Caffeic acid-LG-NH₂ 1% | 0.4 | None |
| Caffeic acid-LG-NH₂ 10% | 0.4 | None |

The test results indicate that Caffeic acid-LG-NH₂ at less than 10% caused no irritation to the skin. Hence, its use as a cosmetic ingredient is considered to cause no skin irritation.

TABLE 4

| | Mean score | Degree of irritation |
|---|---|---|
| Coumaric acid-LG-NH₂ 0.001% | 0.0 | None |
| Coumaric acid-LG-NH₂ 0.01% | 0.0 | None |
| Coumaric acid-LG-NH₂ 0.1% | 0.4 | None |
| Coumaric acid-LG-NH₂ 1% | 0.4 | None |
| Coumaric acid-LG-NH₂ 10% | 0.8 | None |

The test results indicate that Coumaric acid-LG-NH₂ at less than 10% caused no irritation to the skin. Hence, its use as a cosmetic ingredient is considered to cause no skin irritation.

### Experimental Example 5

### Clinical Test for Skin Whitening

Three types of cream compositions were prepared for Comparative example 1 and Examples 1 and 2 according to compositions shown in Table 5 below (unit: % by weight).

TABLE 5

| | Content (% by weight) | | |
|---|---|---|---|
| | Comparative | | |
| Ingredient name | example 1 | Example 1 | Example 2 |
| Caffeic acid-LG-NH₂ | — | 0.1 | — |
| Coumaric acid-LG-NH₂ | — | — | 0.1 |
| Glycerin | 5.0 | 5.0 | 5.0 |
| Stearic acid | 8.0 | 8.0 | 8.0 |
| Squalane | 5.0 | 5.0 | 5.0 |
| Glyceryl monostearate, | 2.5 | 2.5 | 2.5 |
| self-emulsifying type | | | |
| Polyoxyethylene sorbitan | 1.5 | 1.5 | 1.5 |
| monostearate | | | |
| Propylene glycol | 4.0 | 4.0 | 4.0 |
| Stearyl glycyrrhetinate | 0.2 | 0.2 | 0.2 |
| Petrolatum | 2.0 | 2.0 | 2.0 |
| Antioxidant | Moderate | Moderate | Moderate |
| | amount | amount | amount |
| Perfume | Moderate | Moderate | Moderate |
| | amount | amount | amount |
| Preservative | Moderate | Moderate | Moderate |
| | amount | amount | amount |
| Purified water | Balance | Balance | Balance |
| | to 100% | to 100% | to 100% |

A clinical test was conducted to check the skin-whitening effect of creams prepared according to the aforementioned Comparative example 1 and Examples 1 and 2. Thirty women in their 20s to 40s were divided into 3 groups of ten for Comparative example 1 and Examples 1 and 2. They were instructed to use the agents twice per day, after face wash in the morning and after face wash in the evening, for 8 weeks.

A chromameter (Minolta Co., Ltd., Japan) was used to measure skin brightness (L*) before and after use. For comparing effects of treatment, these values were used to obtain ΔL* (whiteness) after 8 weeks of treatment. The results are shown in Table 6.

ΔL*=L*value after 8 weeks of treatment−L*value before treatment Equation 4

TABLE 6

| | ΔL* |
|---|---|
| Comparative example 1 | +2.3 |
| Example 1 | +6.2 |
| Example 2 | +7.6 |

The test results confirmed that 8-week use of Example 1 and 2 containing Caffeic acid-LG-NH₂ and Coumaric acid-LG-NH₂ had greater skin-whitening effect compared to Comparative example 1.

Below is a presentation of several formulation examples using Caffeic acid-LG-NH₂ or Coumaric acid-LG-NH₂ based on the aforementioned test results. However, it should be obvious to a person skilled in the art that these formulation examples are only intended for describing the present invention, and formulations of the invention are not limited to these examples.

### Formulation Example 1

Skin Toner

| Ingredient name | Content (% by weight) |
|---|---|
| Caffeic acid-LG-NH₂ | 0.1 |
| Glycerin | 5.0 |
| 1,3-Butylene glycol | 3.0 |
| Ethanol | 5.0 |
| Polyoxyethylene nonylphenyl ether | 0.5 |
| Perfume | Moderate amount |
| Preservative | Moderate amount |
| Purified water | Balance to 100% |
| Coumaric acid-LG-NH₂ | 0.1 |
| Glycerin | 5.0 |
| 1,3-Butylene glycol | 3.0 |
| Ethanol | 5.0 |
| Polyoxyethylene nonylphenyl ether | 0.5 |
| Perfume | Moderate amount |
| Preservative | Moderate amount |
| Purified water | Balance to 100% |

### Formulation Example 2

### Astringent

| Ingredient name | Content (% by weight) |
|---|---|
| Caffeic acid-LG-NH₂ | 0.1 |
| Glycerin | 3.0 |
| Citric acid | 0.1 |
| Ethanol | 10.0 |
| Polyoxyethylene oleyl ether | 1.0 |
| Sorbitol | 2.0 |
| Perfume | Moderate amount |
| Preservative | Moderate amount |
| Purified water | Balance to 100% |
| Coumaric acid-LG-NH₂ | 0.1 |
| Glycerin | 3.0 |
| Citric acid | 0.1 |
| Ethanol | 10.0 |
| Polyoxyethylene oleyl ether | 1.0 |
| Sorbitol | 2.0 |
| Perfume | Moderate amount |
| Preservative | Moderate amount |
| Purified water | Balance to 100% |

### Formulation Example 3

### Lotion (Emulsion)

| Ingredient name | Content (% by weight) |
|---|---|
| Caffeic acid-LG-NH₂ | 0.1 |
| Glycerin | 5.0 |
| 1,3-Butylene glycol | 8.0 |
| Squalane | 10.0 |
| Polyoxyethylene sorbitan monooleate | 2.0 |
| Triethanolamine | 1.5 |
| Glyceryl stearate | 0.5 |
| Stearyl glycyrrhetinate | 0.2 |
| Carboxyvinyl polymer | 0.1 |
| Arginine | 0.1 |
| Perfume | Moderate amount |
| Preservative | Moderate amount |
| Purified water | Balance to 100% |
| Coumaric acid-LG-NH₂ | 0.1 |
| Glycerin | 5.0 |
| 1,3-Butylene glycol | 8.0 |
| Squalane | 10.0 |
| Polyoxyethylene sorbitan monooleate | 2.0 |
| Triethanolamine | 1.5 |
| Glyceryl stearate | 0.5 |
| Stearyl glycyrrhetinate | 0.2 |
| Carboxyvinyl polymer | 0.1 |
| Arginine | 0.1 |
| Perfume | Moderate amount |
| Preservative | Moderate amount |
| Purified water | Balance to 100% |

### Formulation Example 4

### Essence

| Ingredient name | Content (% by weight) |
|---|---|
| Caffeic acid-LG-NH₂ | 0.1 |
| Sorbitol | 8.0 |
| Polyoxyethylene glycol 1500 | 6.0 |
| Ethanol | 5.0 |
| Glycerin | 3.0 |
| 1,3-Butylene glycol | 3.0 |
| Polyoxyethylene oleyl alcohol ether | 1.0 |
| Olive oil | 0.3 |
| Hyaluronic acid | 0.2 |
| Antioxidant | Moderate amount |
| Perfume | Moderate amount |
| Preservative | Moderate amount |
| Purified water | Balance to 100% |
| Coumaric acid-LG-NH₂ | 0.1 |
| Sorbitol | 8.0 |
| Polyoxyethylene glycol 1500 | 6.0 |
| Ethanol | 5.0 |
| Glycerin | 3.0 |
| 1,3-Butylene glycol | 3.0 |
| Polyoxyethylene oleyl alcohol ether | 1.0 |
| Olive oil | 0.3 |
| Hyaluronic acid | 0.2 |
| Antioxidant | Moderate amount |
| Perfume | Moderate amount |
| Preservative | Moderate amount |
| Purified water | Balance to 100% |

### Formulation Example 5

### Gel

| Ingredient name | Content (% by weight) |
|---|---|
| Caffeic acid-LG-NH₂ | 0.1 |
| Ethanol | 10.0 |
| Glycerin | 4.0 |
| Propylene glycol | 4.0 |
| Polyoxyethylene hydrogenated castor oil | 0.1 |
| Carboxyl polymer | 0.3 |
| Triethanolamine | 0.3 |
| Antioxidant | Moderate amount |
| Perfume | Moderate amount |
| Preservative | Moderate amount |
| Purified water | Balance to 100% |
| Coumaric acid-LG-NH₂ | 0.1 |
| Ethanol | 10.0 |
| Glycerin | 4.0 |
| Propylene glycol | 4.0 |
| Polyoxyethylene hydrogenated castor oil | 0.1 |
| Carboxyl polymer | 0.3 |
| Triethanolamine | 0.3 |
| Antioxidant | Moderate amount |
| Perfume | Moderate amount |
| Preservative | Moderate amount |
| Purified water | Balance to 100% |

### Formulation Example 6

### Ointment

| Ingredient name | Content (% by weight) |
|---|---|
| Caffeic acid-LG-NH₂ | 10.0 |
| Cetostearyl alcohol | 2.0 |
| Monostearic acid, self-emulsifying type | 2.0 |
| Stearic acid | 1.0 |
| Beeswax | 4.0 |
| Squalane | 7.0 |
| Glyceryl monostearate | 3.0 |
| Sorbitan monostearate | 1.0 |
| Polysorbate 80 | 3.0 |
| Glycerin | 5.0 |
| Propylene glycol | 4.0 |
| Perfume | Moderate amount |
| Colorant | Moderate amount |
| Petrolatum | Balance to 100% |
| Coumaric acid-LG-NH₂ | 10.0 |
| Cetostearyl alcohol | 2.0 |
| Monostearic acid, self-emulsifying type | 2.0 |
| Stearic acid | 1.0 |
| Beeswax | 4.0 |
| Squalane | 7.0 |
| Glyceryl monostearate | 3.0 |
| Sorbitan monostearate | 1.0 |
| Polysorbate 80 | 3.0 |
| Glycerin | 5.0 |
| Propylene glycol | 4.0 |
| Perfume | Moderate amount |
| Colorant | Moderate amount |
| Petrolatum | Balance to 100% |

1. A cosmetic composition for skin whitening comprising, as an active ingredient, chemical compounds expressed in the following Formula 1: 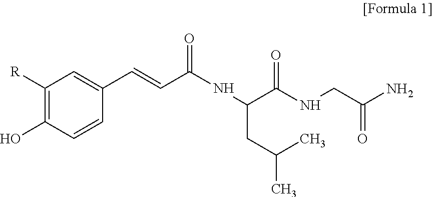
wherein R is H or OH.

2. The cosmetic composition for skin whitening according to claim 1 wherein the chemical compounds are contained 0.000001 to 30% by weight with respect to the total weight of the cosmetic composition.

3. The cosmetic composition for skin whitening according to claim 1 wherein the formulation of the skin-whitening cosmetic composition is for skin external use.

4. The cosmetic composition for skin whitening according to claim 1 wherein the formulation of the skin-whitening cosmetic composition is selected from a group consisting of fluid, gel, serum, cream, essence, oil-in-water emulsion, water-in-oil emulsion, ointment, and a combination thereof.

5. The cosmetic composition for skin whitening according to claim 1, further comprising an additive selected from a group consisting of oil, water, surfactants, moisturizers, lower alcohols, thickeners, chelation agents, colorants, preservatives, perfumes, and a mixture thereof.
